(19) United States
(12) Patent Application Publication
(10) Pub. No.: US 2023/0092983 A1
(43) Pub. Date: Mar. 23, 2023

(54) Systems and Methods for Managing Brain Injury and Malfunction

(71) Applicant: Power of Patients, LLC, Boston, MA (US)

(72) Inventor: Lynne E. Becker, Charlestown, MA (US)

(73) Assignee: Power of Patients, LLC, Boston, MA (US)

(21) Appl. No.: 17/795,267

(22) PCT Filed: Sep. 12, 2021
(86) PCT No.: PCT/US21/49983

§ 371 Date: Jul. 26, 2022

Related U.S. Application Data
(60) Provisional application No. 63/077,323, filed on Sep. 11, 2020.

(51) Int. Cl. A61B 5/00

(52) U.S. Cl. CPC A61B 5/4064; A61B 5/7275; A61B 5/7475; A61B 5/742; A61B 5/746

(57) ABSTRACT

Systems and methods for managing brain injury or brain malfunction involve receiving symptoms, assessments, social determinants of health, and substrates for evaluation, correlating that data to identify triggers, boosts, and changes in the patient, and notifying the patient or her caregiver of those triggers, boosts, and changes, in some cases via a dashboard display. Triggers cause changes for the worse for the patient, while boosts cause changes for the better. Certain instances marshal enormous amounts of evidence never correlated before in the recovery from trauma to the brain.

### CROSS-REFERENCE TO RELATED APPLICATIONS

This application claims benefit of priority under PCT Chapter I, Article 8, and 35 U.S.C. § 119(e) of U.S. Provisional Patent Application No. 63/077,323 entitled, “SYSTEMS AND METHOD FOR MANAGING BRAIN INJURY AND MALFUNCTION,” filed on Sep. 11, 2020, which is incorporated herein by reference in its entirety.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### FIELD OF INVENTION

This invention relates to machine learning in the art of managing a patient's recovery from brain injury or brain malfunction.

### BACKGROUND OF THE INVENTION

Little is truly known about brain injury and brain malfunction. Severe concussions sometimes cause no apparent lasting effects, while minor head injuries often change the life of the victims. A subsequent blow to the head while a patient recovers from an earlier trauma may have an effect far out of proportion to the magnitude of the blow. Many patients endure symptoms exacerbated by unknown triggers, magnifying suffering while delaying recovery and return to normal functionality. Strokes rob patients of memories, talents, and functionalities from basic life skills to complex abilities honed over a lifetime and almost taken for granted before their loss. Brain injuries formally classified as traumatic brain injuries (“TBIs”), Parkinson's disease, Alzheimer's disease, other forms of dementia, and brain tumors, among other infirmities, each attack a patient by afflicting the brain. Even life-prolonging treatments such as chemotherapy and radiation therapy may cause brain fog and require the brain to recover. Trauma to the brain carries with it profound impacts on a person's physical health, social wellbeing, and economic productivity dramatically more so than trauma to any other part of the body.

Yet diagnosis and treatment of brain trauma remains rudimentary. So often any treatment protocol is driven by compensation-directing codes such as Current Procedural Terminology or CPT® codes, or by International Statistical Classification of Diseases and Related Health Problems, or ICD codes, promulgated by the World Health Organization. If a code does not exist or is not easily found for the nuanced constellation of symptoms a patient suffers, the best the patient can hope for is a square peg in a round hole kind of diagnosis and treatment. Rest is most often favored once brain trauma is suspected. Measurable influences on recovery and exacerbation of symptoms go ignored or remain unknown, because the tools to record and study those influences do not exist. It is known, for example, to measure eye-hand coordination, or eye movements, to diagnose brain trauma. Yet nothing currently exists to determine why, for example, eye-hand coordination suddenly declines in a patient six months after an initial injury. Medical practitioners have no disciplined way to quantitatively monitor characteristics such as eye-hand coordination over time, let alone determine what might influence changes for the worse or for the better in such characteristics. Further, neither medical practitioners nor patients have any tool whatsoever to compare a successful treatment regimen in one patient to the proposed treatment for a newly-injured or still-recovering patient suffering under similar circumstances. Better tools are needed to help patients and their caregivers monitor recovery from various types of brain trauma.

### SUMMARY OF THE INVENTION

Unexpectedly, Applicant has developed tools for managing brain trauma recovery. This development stems from, among other activity, the inventor acting as caregiver and patient advocate for family members suffering from diverse severe brain traumas. In some instances, embodiments of the present invention may provide the patient or her caregiver patient-centric tools to record, monitor, and correlate seemingly unrelated resurgence of suffering with those environmental inputs that drive that resurgence. Similarly, other instances use those patient-centric tools to correlate seemingly unrelated improvement with those environmental inputs that drive that improvement. Accordingly, some embodiments of the present invention relate to systems for managing brain injury or brain malfunction in a patient in need thereof, one such system comprising:

an input database configured to receive

symptoms,

assessments,

social determinants of health, and

substrates for evaluation;

a correlation engine configured to
(a) process the substrates for evaluation from the input database to obtain observables;
(b) correlate the symptoms, the assessments, and the social determinants of health from the input database, and the observables, to determine at least one trigger, at least one boost, or both;
(c) detect a change for the worse or a change for the better in any of the symptoms, the assessments, the social determinants of health, or the observables greater than one or more thresholds over time; and
(d) indicate that one or more of additional symptoms, additional assessments, additional social determinants of health, and additional substrates for evaluation are needed;
a communication engine configured to
(i) reach out to the patient or to a caregiver thereof to seek input of the symptoms, the assessments, the social determinants of health, the substrates for evaluation, the additional symptoms, the additional assessments, the additional social determinants of health, and the additional substrates for evaluation;
(ii) guide the patient or the caregiver to input the symptoms, the assessments, the social determinants of health, the substrates for evaluation, the additional symptoms, the additional assessments, the additional social determinants of health, and/or the additional substrates for evaluation to the input database,
wherein the additional symptoms, the additional assessments, the additional social determinants of health, and the additional substrates for evaluation, once inputted into the input database, are treated the same as the symptoms, the assessments, the social determinants of health, and the substrates for evaluation, respectively;
   (iii) warn the patient or the caregiver when the change for the worse in any of the symptoms, the assessments, the social determinants of health, or the observables greater than one or more thresholds over time has been detected by the correlation engine;
   (iv) encourage the patient or the caregiver when the change for the better in any of the symptoms, the assessments, the social determinants of health, or the observables for the better greater than one or more thresholds over time has been detected by the correlation engine; and
   a dashboard engine configured to graphically display the symptoms, the assessments, the social determinants of health, the substrates for evaluation, the observables, the triggers, and/or the boosts,
   thereby managing the brain injury or brain malfunction in the patient.

Other embodiments of the present invention relate to methods for managing brain injury or brain malfunction in a patient in need thereof, one such method comprising:

receiving in memory

symptoms,

assessments,

social determinants of health, and

substrates for evaluation;

processing, using a processor, the substrates for evaluation to obtain observables; correlating, using the processor, the symptoms, the assessments, the social determinants of health, and the observables, to determine at least one trigger, at least one boost, or both;
detecting, using the processor, a change for the worse or a change for the better in any of the symptoms, the assessments, the social determinants of health, or the observables greater than one or more thresholds over time;
indicating, using the processor, that one or more of additional symptoms, additional assessments, additional social determinants of health, and additional substrates for evaluation are needed;
reaching out, using the processor, to the patient or to a caregiver thereof to seek input of the symptoms, the assessments, the social determinants of health, the substrates for evaluation, the additional symptoms, the additional assessments, the additional social determinants of health, and the additional substrates for evaluation;
guiding, using the processor, the patient or the caregiver to input the symptoms, the assessments, the social determinants of health, the substrates for evaluation, the additional symptoms, the additional assessments, the additional social determinants of health, and/or the additional substrates for evaluation into memory,
wherein the additional symptoms, the additional assessments, the additional social determinants of health, and the additional substrates for evaluation, once received by memory, are treated the same as the symptoms, the assessments, the social determinants of health, and the substrates for evaluation, respectively;
   warning, using the processor, the patient or the caregiver when the change for the worse in any of the symptoms, the assessments, the social determinants of health, or the observables greater than one or more thresholds over time has been detected by the processor;
   encouraging, using the processor, the patient or the caregiver when the change for the better in any of the symptoms, the assessments, the social determinants of health, or the observables greater than one or more thresholds over time has been detected by the processor;
   graphically displaying, using the processor, the symptoms, the assessments, the social determinants of health, the substrates for evaluation, the observables, the triggers, and/or the boosts, thereby managing the brain injury or brain malfunction in the patient.

Still further embodiments relate to non-transitory computer program products, one such product comprising a program code that, upon execution by a processor, is configured to perform a method for managing brain injury or brain malfunction in a patient in need thereof, comprising:

receiving in memory

symptoms,

assessments,

social determinants of health, and

substrates for evaluation;

processing, using a processor, the substrates for evaluation to obtain observables; correlating, using the processor, the symptoms, the assessments, the social determinants of health, and the observables, to determine at least one trigger, at least one boost, or both;
detecting, using the processor, a change for the worse or a change for the better in any of the symptoms, the assessments, the social determinants of health, or the observables greater than one or more thresholds over time;
indicating, using the processor, that one or more of additional symptoms, additional assessments, additional social determinants of health, and additional substrates for evaluation are needed;
reaching out, using the processor, to the patient or to a caregiver thereof to seek input of the symptoms, the assessments, the social determinants of health, the substrates for evaluation, the additional symptoms, the additional assessments, the additional social determinants of health, and the additional substrates for evaluation;
guiding, using the processor, the patient or the caregiver to input the symptoms, the assessments, the social determinants of health, the substrates for evaluation, the additional symptoms, the additional assessments, the additional social determinants of health, and/or the additional substrates for evaluation into memory,
wherein the additional symptoms, the additional assessments, the additional social determinants of health, and the additional substrates for evaluation, once received by memory, are treated the same as the symptoms, the assessments, the social determinants of health, and the substrates for evaluation, respectively;
   warning, using the processor, the patient or the caregiver when the change for the worse in any of the symptoms, the assessments, the social determinants of health, or the observables greater than one or more thresholds over time has been detected by the processor;
   encouraging, using the processor, the patient or the caregiver when the change for the better in any of the symptoms, the assessments, the social determinants of health, or the observables greater than one or more thresholds over time has been detected by the processor;
   graphically displaying, using the processor, the symptoms, the assessments, the social determinants of health, the substrates for evaluation, the observables, the triggers, and/or the boosts, thereby managing the brain injury or brain malfunction in the patient.

Still further embodiments relate to methods of training a system for managing brain injury or brain malfunction in a patient in need thereof, one such method comprising providing the system with an initial dataset comprising one or more of initial symptoms, initial assessments, initial social determinants of health, initial substrates for evaluation, initial observables, and initial anomalies; and identifying for the system initial triggers comprising negative correlations between one or more of the initial symptoms, the initial assessments, the initial social determinants of health, the initial substrates for evaluation, the initial observables, and the initial anomalies, and identifying for the system initial boosts comprising positive correlations between one or more of the initial symptoms, the initial assessments, the initial social determinants of health, the initial substrates for evaluation, the initial observables, and the initial anomalies, thereby training the system.

Additional embodiments relate to a system optionally comprising a neural network trained to identify triggers in a patient suffering from brain injury or brain malfunction, comprising a pre-populated library populated with one or more of initial symptoms, initial assessments, initial social determinants of health, initial substrates for evaluation, initial observables, initial anomalies, initial triggers, and initial boosts. Similarly, further additional embodiments relate to a system comprising a neural network trained to identify boosts in a patient suffering from brain injury or brain malfunction, comprising a pre-populated library populated with one or more of initial symptoms, initial assessments, initial social determinants of health, initial substrates for evaluation, initial observables, initial anomalies, initial triggers, and initial boosts. Libraries can be populated or pre-populated from the perspective of performing a given method according to any suitable protocol. In some cases, a body of information relating to specific patients in a specific population can be imported and organized for the use of a system described herein.

Yet other additional embodiments provide a library for a system for managing brain injury or brain malfunction in a patient in need thereof, comprising one or more of symptoms, assessments, social determinants of health, substrates for evaluation, observables, and anomalies, optionally triggers, and optionally boosts.

Certain further instances of the present invention relate to a system trained to identify triggers in a patient recovering from brain injury or brain malfunction, wherein the system identifies the triggers from correlations between one or more symptoms, one or more assessments, one or more social determinants of health, one or more substrates for evaluation, one or more observables, one or more anomalies, or a combination thereof. Yet additional instances provide a system trained to identify boosts in a patient recovering from brain injury or brain malfunction, wherein the system identifies the boosts from correlations between one or more symptoms, one or more assessments, one or more social determinants of health, one or more substrates for evaluation, one or more observables, one or more anomalies, or a combination thereof.

Still other instances relate to a system trained to predict one or more symptoms in a patient recovering from brain injury or brain malfunction, wherein the system predicts the one or more symptoms having been trained on an initial dataset comprising one or more of initial symptoms, initial assessments, initial social determinants of health, initial substrates for evaluation, initial observables, and initial anomalies.

While the disclosure provides certain specific embodiments, the invention is not limited to those embodiments. A person of ordinary skill will appreciate from the description herein that modifications can be made to the described embodiments and therefore that the specification is broader in scope than the described embodiments. All examples are therefore non-limiting.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 schematically depicts one embodiment of the invention comprising system 100.

FIG. 2 schematically depicts components of system 100 in one possible configuration.

FIG. 3 provides a flowchart of method 300.

FIG. 4 provide a flowchart of method 400.

FIG. 5 provide a flowchart of method 500.

FIG. 6 shows a mockup screenshot of graphical user interface 600.

FIG. 7 shows a mockup screenshot of graphical user interface 700.

FIG. 8 shows a mockup screenshot of graphical user interface 800.

FIG. 9 depicts mockup graph 900.

FIG. 10 depicts table 1000.

FIG. 11 schematically depicts system 1100.

FIG. 12 shows code 1200 suitable for training a system using a Random Forest Classifier.

FIG. 13 shows code 1300 suitable for predicting symptoms for patients having entered social determinants of health.

FIG. 14 shows code 1400 suitable for predicting symptoms for patients having entered a written narrative as a substrate for evaluation.

### DETAILED DESCRIPTION

As required, detailed embodiments of the present invention are disclosed herein; however, it is to be understood that the disclosed embodiments are merely exemplary of the invention that may be embodied in various forms. The figures are not necessarily to scale, and some features may be exaggerated to show details of particular components. Therefore, specific structural and functional details disclosed herein are not to be interpreted as limiting, but merely as a basis for the claims and as a representative basis for teaching one skilled in the art to variously employ the present invention.

Unless defined otherwise, all technical and scientific terms used herein have the same meaning as is commonly understood by one of ordinary skill in the art to which this disclosure belongs. In the event that there is a plurality of definitions for a term herein, those in this disclosure prevail unless stated otherwise.

As used herein, it is not important to distinguish between “brain injury” and “brain malfunction.” “Brain injury” refers to physical damage to the brain, such as that caused by a blow to the head, or ischemic or hemorrhagic injury caused by an embolism or bleeding from a blood vessel in the brain, a stroke. “Brain malfunction” refers to the loss of function. That loss of function may be apparently related to an injury such as a blow or a stroke, but sometimes loss of function is not apparently related to an injury. For example, a patient suffering from an injury following a physical blow may experience a malfunction, such as, for example, a loss of words or memory. For another example, a patient recovering from a brain injury may experience a loss of function months after the injury, and the loss is not apparently related to the injury because of the passage of time. In yet another example, a patient suffering from Alzheimer's disease, Parkinson's disease, or general dementia suffers brain malfunction without a readily-identifiable brain injury. Still further examples include chemotherapy or radiation patients enduring brain fog—a brain malfunction caused by the “injury” of therapy. Genetic predispositions also could benefit from a careful look at environmental factors influencing brain malfunction. Both “brain injury” and “brain malfunction” may be referred to as “brain trauma” herein.

As used herein, “negative” and “positive” are used to indicate worsening or improving conditions for the patient, respectively. Thus, a “negative correlation” indicates a relationship between environmental factors and a worsening condition for the patient. A “positive correlation” indicates a relationship between environmental factors and an improving condition for the patient. In other words, a “negative correlation” means a correlation that is negative or undesirable for the patient. A “positive correlation” indicates a correlation that is positive or desirable for the patient.

As used herein, “caregiver” can mean any person involved in the patient's wellbeing. In some instances, a caregiver is a close family member, such as a parent looking after a child patient. In other instances, a caregiver is a medical professional. In still other instances, a close family member and medical professionals can participate with the systems and methods described herein as caregivers. A “medical professional” is not limited, and includes, for example, a case manager, a primary care physician, specialist physicians, physician assistants, nurses, nurse practitioners, technicians, social workers, physical therapists, vestibular therapists, occupational therapists, speech therapists, opthalmologists, optometrists, even therapy dog handlers.

Wherever the phrase “for example,” “such as,” “including” and the like are used herein, the phrase “and without limitation” is understood to follow unless explicitly stated otherwise. Similarly “an example,” “exemplary” and the like are understood to be non-limiting.

The term “substantially” allows for deviations from the descriptor that don't negatively impact the intended purpose. Descriptive terms are understood to be modified by the term “substantially” even if the word “substantially” is not explicitly recited.

The term “about” when used in connection with a numerical value refers to the actual given value, and to the approximation to such given value that would reasonably be inferred by one of ordinary skill in the art, including approximations due to the experimental and or measurement conditions for such given value.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a device having components a, b, and c” means that the device includes at least components a, b and c. Similarly, the phrase: “a method involving steps a, b, and c” means that the method includes at least steps a, b, and c.

Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

Any discussion of the prior art throughout the specification should in no way be considered as an admission that such prior art is widely known or forms part of common general knowledge in the field.

It is an object of the present invention to overcome or ameliorate at least one of the disadvantages of the prior art, or to provide a useful alternative.

As mentioned, one embodiment of the present invention relates to a system for managing brain injury or brain malfunction in a patient in need thereof. Such a system can include any suitable memory or storage structures, including an input database configured to receive symptoms, assessments, social determinants of health, and substrates for evaluation. Such data can come from any suitable source. In some cases, a patient can provide such data. In other cases, a caregiver provides such data. In still other cases, the system can reach out to other sources of information. For example, if the patient or her caregiver have recorded the patient's zip code in a patient profile associated with the patient, the system can be configured to obtain weather information for that zip code for any given date. As can be appreciated, weather conditions may have an impact on a patient's health, and information about weather conditions can be obtained reliably from weather information databases available on the internet. Accordingly, some embodiments of the present invention allow for the system to receive, create, store, or a combination thereof, a patient profile. A patient profile can have any suitable information. Name, date of birth, gender, sex, race, medical history other than brain trauma, brain traumatic events including the nature of any injury, where on the head or neck any impact may have been received, how deep in the brain the injury may have impacted, date of brain traumatic events, treatment since any brain traumatic events, and the like may be included in a profile. In some cases, data from the patient profile can be treated by the system as social determinants of health as desired.

The system can be configured to receive information such as the symptoms, the assessments, the social determinants of health, and the substrates for evaluation, in any suitable manner. In some cases, the communication engine, optionally working with the dashboard engine, can guide the patient or her caregiver to input data in a numerical or other form that requires no further processing. In other cases, the patient or her caregiver can input text or speech that requires natural language processing to obtain the data in a form the system can use for correlation, for example. Any suitable natural language (“NPL”) processing protocol can be used. In certain instances, an NPL application programming interface (“API”) can be accessed, such as, for example, Aylien text API, IBM Watson Alchemy API, Microsoft Text Analytics API, and Linguakit API.

Any suitable symptom can be recorded by the system. Sleep problems, emotional problems, cognitive problems, and physical symptoms may be mentioned. In some cases, the symptoms comprise one or more of general wellbeing, headache, migraines, nausea, dizziness, fatigue, excessive sleeping, difficulty sleeping, little sleep, difficulty falling asleep, anxiety increasing at bedtime, drowsiness, sleep apnea, narcolepsy, loss of vocabulary, slurred speech, sensitivity to light, sensitivity to sound, forgetfulness, difficulty concentrating, changes in mood, anxiousness, depression, unexplained sadness or crying, loss of motivation, irritability, suicidal ideation, brain fog, dyslexia, stuttering, attention deficit hyperactivity disorder (ADHD), short term memory loss, long term memory loss, repeating oneself, loss of appetite, constant hunger, weight gain, weight loss, loss of or change in taste, loss of or change in smell, loss or lack of coordination, dropping objects, ringing in ears, blurred vision, paralysis or partial paralysis, and seizures. A patient can be asked to track any suitable number of symptoms. For example, a patient can be asked to track one, two, three, four, five, six, seven, eight, nine, or ten symptoms over time. In another example, a patient is asked to track three symptoms over time.

For convenience, or to assist the patient or her caregiver to focus on certain areas, symptoms can be organized in any suitable fashion. In some cases, symptoms can be organized by conditions, such as, for example, cognitive conditions, sleep conditions, emotional conditions, physical conditions, speech pathology conditions, and visual conditions, for example. It is not critical whether a given symptom appears in a given group of conditions. For example, light sensitivity can appear among physical conditions, vision conditions, or both. To illustrate, cognitive conditions can include, for example, brain fog, lack of focus, short term memory loss, poor concentration, slow thinking or processing, can't find the right words, dyslexia, stuttering, and long-term memory loss. Sleep conditions can include, for example, excessive sleep, poor or little sleep, fatigue, excessive exhaustion, extreme yawning, drowsiness, narcolepsy, sleep apnea, words jump, tremors in hands, constipation, extremities of toes and hands often cold, and shaking. Emotional conditions may include, for example, depression, anxiety, mood swings, unexplained sadness/crying, no motivation, anger, very nervous, panic attacks, irritability, and impulsiveness. Physical conditions may include, for example, headaches and/or migraines, loss of balance/dizziness, loss of smell, light sensitivity, ringing in ears, noise sensitivity, vomiting/digestive issues, heart sensations, slurred speech, lack of coordination, unexplained dropping objects, loss of hand control, repeating oneself, loss of appetite, always hungry, and loss of taste. Speech pathology conditions may include, for example, trouble staying organized, trouble managing daily tasks, less responsive to the environment, limited social engagement, difficulty expressing needs, limited communication, trouble remembering names, challenging to count to ten, struggles to multitask, trouble with self-discipline, difficulty understanding abstract ideas, and difficulty planning in advance. Visual conditions include, for example, blurred vision, uncomfortable eyes, double vision, bothered by light, trouble perceiving depth, distorted side vision, dry eyes, irritated by visually-busy places, words move when reading, chronic itchiness, line skipping when reading, eyes fatigue quickly or easily, headaches caused by close work: computers, reading, gaming. Optionally, within a group of conditions or otherwise, the patient or her caregiver may be offered the option to input other symptoms in the patient's or caregiver's own words.

Any suitable assessments can be received from the patient or the caregiver. For example, suitable assessments comprise the patient or the caregiver assigning a numerical value to one or more of mood, quality of sleep, quantity of sleep, level of appetite, ability to concentrate, mental function, emotional function, sociability, physical activity, and quality of life. Optionally, the assessments can be tallied together to obtain an overall score. For example, the system can prompt the patient to enter numerical values for “poor concentration,” “excessive sleeping,” and “unexplained sadness/crying,” and the sum of those assessments can inform whether an overall improvement or decline can be perceived, even if those assessments vary greatly. A symptom and an assessment differ in this manner: a symptom is a qualitative fact, while an assessment provides a numerical value. “I don't feel like eating” is an expression of the symptom of loss of appetite, while a numerical value that the patient assigns to the strength of her appetite would be called an assessment.

Suitable social determinants of health include, but are not limited to, one or more of weather, outside temperature, inside temperature, transportation, physical activity, exercise, social activity, financial well-being, race, color, religion, and gender. Exposure to bright sunshine or bright light; exposure to loud sounds; diet, including nutritional supplements, medicines, amounts of fats, protein, carbohydrates, sugar, alcohol, water, and/or caffeine consumed; dehydration; fasting; altitude, and recent changes in altitude including air travel or ground travel resulting in altitude changes; latitude changes; time zone changes; menstrual cycle; stress, among other factors, can be included as a social determinant of health. The answers to the questions, “where on your head were you hit?” and “how were you injured” can be treated as social determinants of health, or optionally as a symptom. Broadly speaking, “social determinants of health” can be thought of as a catch-all category. Risk factors such as family history, family medical history, personal history, personal medical history, occupation, toxin exposure, exercise habituation, leisure activities, recent activities, therapies received, and the details thereof all can be collected as social determinants of health. It is unknown, in some cases, what causes a worsening or an improvement in a patient; no information is categorically excluded from certain embodiments of the present invention. Social determinants of health can be grouped together, such as, for example, into wellness determinants, travel determinants, screen time determinants, environmental determinants, and other determinants. It is not critical whether a particular determinant appears in one group or another.

The system can be configured to provide any suitable access to caregivers. In some cases, a system is configured to allow access only for a patient and her close family member serving as a caregiver. In other cases, one or more medical professionals also can be granted access. That access can be staged as desired in any suitable fashion. All caregivers could have the same level of access, for example, or different caregivers could have different levels of access. A case manager or lead physician could have comprehensive access to review, to input, and to schedule future prompts, in some instances. In other instances, a therapeutic dog handler would have access only sufficient to log a visit with the patient by a therapy dog, for example.

The system would have a processor. As used herein, “processor” includes a single processor or multiple processors working together. Such processors can form the core of a single machine such as a multi-processor CPU, or such processors can be distributed across a network. Any suitable processors can be included in the systems of the present invention.

The processor can be configured to process the substrates for evaluation from the input database to obtain observables. In practice, this means the processor and subordinate components are configured to perform one or more of a variety of tasks. Any suitable substrate for evaluation can be included. Medical data such as QEEG data, CAT scans, MRI scans, functional MRI scans, ultrasound images, and other such data can provide substrates for evaluation. Data from purpose-designed devices or software operating on a general-purpose computer also can provide substrates for evaluation. For example, Cognivue, Inc., provides several FDA-approved platforms for assessing a patient's cognitive function. Those platforms yield reports on the patient's performance, which can provide substrates for evaluation. A patient manipulating a mouse while tracking images on a computer screen also may yield data as a substrate for evaluation. A doctor-prescribed “homework” can also provide a substrate for evaluation, optionally yielding symptoms and social determinants of health, as well. A written narrative provided by the patient can be graded according to any suitable standard. For example, the written narrative can be evaluated for numerous common grammatical errors, such as, for instance, lack of punctuation and capitalization, run-on sentences, misuse of homophones (e.g., there, their, and they're), common spelling mistakes of simple words, and common spelling mistakes of complex words. A spoken narrative also can be evaluated, for pace, tone, inflection, sentence fragments, slurred speech, complexity of sentence structure, range of vocabulary such as number of unique words, loss of words, and loss of train of thought, for example. Natural language processing algorithms and tools, such as are known in the art, can be employed to recognize the words and sentences of the spoken narrative and then to grade it. Grades can be assigned based on a corresponding scholastic grade level, or on an arbitrary scale of 1 to 100, in certain cases.

Various APIs exist and are known for the grading of written text. Any suitable API can be used. The Automated Readability Index, Coleman-Liau Index, Gunning Fog Index, SMOG Index, and Flesch-Kincaid Grade Level Index may be mentioned for determining the scholastic grade level of a patient's written narrative. However, Applicant has found that at least some of those APIs may be inapplicable if a patient's narrative is very poorly written, as may be the case for a patient suffering from severe brain trauma. Other APIs such as Fry Index, Raygor Index, and Cloze Test, are available at www.analyzemywriting.com (accessed Aug. 26, 2020).

In further cases, one or more images of the patient can provide substrates for evaluation. Unexpectedly, Applicant has found that asymmetry in the eyelids of a patient indicates a flare-up of symptoms in patients who have suffered traumatic brain injury. Also, asymmetry in the dilation of the pupils may further indicate a flare-up of symptoms. These asymmetries can be called “anomalies,” and represent observables when present. Such images can come from any suitable source, including the patient's own smart phone.

In still further cases, so-called brain games can provide substrates for evaluation. As used herein, a brain game is any task or series of tasks for the patient to perform that can reveal the mental abilities of the patient. In some cases, a brain game reveals a binary, yes-or-no result, while in other cases, a brain game is designed to reveal relative ability. For example, the patient may be asked to add two numbers together. The patient's performance on this task is binary: either the patient is able to get the right result, or she is not. In another brain game, the patient may be asked to memorize ten numbers. The greater number of memorized numbers correctly repeated reveals a greater mental ability. A patient correctly memorizing eight numbers has a greater short-term memory ability than a patient correctly memorizing only two.

In some embodiments, brain games can test the interaction between the eyes, the hands, and different parts of the brain. In the so-called Mocha Test, a patient is asked to trace, in alphabetical order, a series of letters randomly appearing on a page. Such a test can be adapted to appear on a screen as well. The speed at which the task is completed and also whether any mistakes are made can be recorded as observables. It is further possible to create brain games of increasing complexity. For example, the patient can be asked to find a particular character, such as a zero or an “A.” In a simple level, numerous characters can appear in a single color against a contrasting background, such as black characters on a white screen. On more complex levels, color combinations, characters with fuzzy or out-of-focus edges, and characters overlapping with each other can be introduced. At an even more complex level, motion can be introduced, in which characters move around the screen. The speed of the response and ability to recognize given characters can provide observables, and changes in those observables can be noted over days, weeks, or months. Similarly, a game such as video ping pong can test hand-eye coordination, and speed of the digital ball and score can be recorded as observables.

The system can be configured to correlate the symptoms, the assessments, and the social determinants of health from the input database, and the observables, to determine at least one trigger, at least one boost, or both. In practice, correlation can happen according to any suitable protocol. In some cases, correlation may simply look for common occurrence of two inputs, such as, for example, whether (a) a patient has received a blow on the left side of the head and (b) experiences poor concentration (a symptom) or assigns a high number to “poor concentration” (an assessment). In other cases, a sudden-onset symptom or a change in any of the symptoms, assessments, social determinants of health, or observables can represent a landmark in time for the system to orient its correlation. From that landmark, the system iteratively looks backward in time to identify any events that could be relevant to that landmark. The system can then look for similar landmarks, and iteratively look backward in time seeking any events relevant to the similar landmarks. If a similar event appears before similar landmarks, a correlation may be established and a trigger or a boost identified. For example, if a patient records severe headaches occurring from time to time, it may be discovered that significant vehicular travel one to three days ahead of onset of the headaches correlates to the headaches, that is, that vehicular travel triggers the headaches. The system would record vehicular travel as a trigger. In some cases, it can be said that a trigger represents a relationship between at least one of the social determinants of health and a worsening of at least one of the symptoms, at least one of the assessments, at least one of the observables, or a combination thereof. In further cases, a trigger represents a relationship between a significant change for the worse, i.e., a change greater than a pre-determined threshold, in at least one of the social determinants of health and a significant change in at least one symptom, a significant change in at least one assessment, a change in at least one observable, or a combination thereof. Once identified, a trigger can be presented to the patient or the caregiver in any suitable fashion. For example, the communication engine and/or the dashboard engine can be tasked with encouraging the patient and the caregiver to minimize, avoid, or otherwise manage the trigger.

Conversely, a boost represents a relationship between at least one of the social determinants of health and an improvement of at least one of the symptoms, at least one of the assessments, at least one of the observables, or a combination thereof. In further cases, a boost represents a relationship between a significant change for the better, i.e., a change greater than a pre-determined threshold, in at least one of the social determinants of health and a significant change in at least one symptom, a significant change in at least one assessment, a change in at least one observable, or a combination thereof. Once identified, a boost can be presented to the patient or the caregiver in any suitable fashion. For example, the communication engine and/or the dashboard engine can be tasked with encouraging the patient and the caregiver to maximize, pursue, or otherwise manage the boost. A boost can also relate to the improvement of brain function. For example, soothing music or resting in a darkened room may lessen headaches for a patient, and thereby represent boosts for the patient. In that way, a boost is an environmental factor that lessens an undesirable symptom. But a boost also can represent an improvement where there is no symptom. If a patient routinely experiences greater short-term memory ability after a day of exercise followed by a good night's sleep, the exercise and sleep could be boosts for that patient, even if short-term memory loss or lessened ability are not symptoms for that patient.

In still other embodiments, the system can be trained to find correlations between symptoms, assessments, social determinants of health, and substrates for evaluation, observables, and anomalies, and identify triggers and boosts thereby. Any suitable model can be used to train the system. Random Forest Classifier, Neural Net, K-Nearest Neighbors, and Decision Tree Classifier may be mentioned. In some cases, a patient or her caregiver can record social determinants of health, for example, and the trained system can predict the symptoms the patient will experience. This has value in preparing the patient for upcoming symptoms, and also allowing the patient to customize the system to the patient by confirming, refuting, and refining the system's predictions.

The system also may be configured to record changes in any of the symptoms, the assessments, the social determinants of health, or the observables greater than one or more thresholds over time. Any suitable threshold may be chosen. In some embodiments, thresholds represent a change of greater than about 5%, greater than about 10%, greater than about 20%, greater than about 25%, greater than about 30%, greater than about 40%, greater than about 50%, greater than about 75%, greater than about 90%, or greater than about 100%. Such changes can move in either direction—worse for the patient, or better for the patient. When the change is worse for the patient, a trigger may be involved. If the change is for the better, a boost may be the cause. Any suitable time period may be chosen. A day or days, a week or weeks, a month or months may be mentioned. In some cases, a change of 30% in a day can be identified as a change greater than the threshold and reported as described herein. In some cases, the system can be configured to record the absence of changes in any given symptom, assessments, social determinants of health, or observables—a plateau—over a given period of time. Any suitable plateau can be defined, such as, for example, a plateau of one month, two months, three months, four months, five months, or six months. A plateau can be further defined by any suitable lack of change, such as, for example, a change no greater than about 5%, no greater than about 10%, no greater than about 20%, no greater than about 25%, no greater than about 30%, no greater than about 40%, no greater than about 50%, no greater than about 75%, no greater than about 90%, or no greater than about 100%.

In some cases, the system is configured to indicate that one or more of additional symptoms, additional assessments, additional social determinants of health, and additional substrates for evaluation are needed. This need can be identified in any suitable manner. In some cases, the system can be programmed to identify that need if the patient or caregiver has not logged into the system within the last day, the last week, or any suitable period. In other cases, the system can identify the need for additional data by noting a lack of a particular kind of data for a given period. For example, a patient may be diligent about entering symptoms but skipping assessments. The system would record a need for the assessments. In still other cases, the system can identify the need for more of a particular kind of data. For example, if the system identifies a recurring symptom such as severe headaches and correlates vehicular travel as a potential trigger for some occurrences for those headaches but not others, the system could identify the need to inquire whether vehicular travel occurred before those other occurrences. In yet additional cases, the system could detect a significant change for the better and prompt the patient and/or the caregiver to provide more information to aid the hunt for the boost or boosts that may have caused that change for the better.

A system comprises a communication engine. Any suitable communication engine can be used. In some cases, the communication engine is configured to reach out to the patient or to a caregiver thereof to seek input of the symptoms, the assessments, the social determinants of health, the substrates for evaluation, the additional symptoms, the additional assessments, the additional social determinants of health, and the additional substrates for evaluation. For clarity, it can be said that input is sought for a “symptom,” for example, when no data of that symptom has been received. Input is sought for an “additional symptom” when some data of that symptom has been received, but the system identifies a need for more data of that symptom, if any. Once entered into the input database, the additional symptoms, the additional assessments, the additional social determinants of health, and the additional substrates for evaluation, are treated the same as the symptoms, the assessments, the social determinants of health, and the substrates for evaluation, respectively. As used herein, “reaching out” can include any suitable technological communication mode. In some cases, reaching out to the patient or the caregiver comprises the system sending to the patient and/or the caregiver at least one electronic communication chosen from a text message, an email message, an automated phone call, or a combination thereof. In further cases, reaching out to the patient or the caregiver comprises sending, using the processor, a prompt to a medical care professional to call or email or otherwise message the patient or the caregiver, to add a personal touch to the care of the patient. This reaching out can be done even if, or especially if, the patient or caregiver is not logged into the system. In that way, the system can provide a powerful tool for assisting a patient suffering from a traumatic brain injury or other malfunction of the brain to manage recovery. Such a system would not rely on the patient's own volition to engage the system, which could be lacking.

The communication engine can be configured further to guide the patient or the caregiver to input the symptoms, the assessments, the social determinants of health, the substrates for evaluation, the additional symptoms, the additional assessments, the additional social determinants of health, and/or the additional substrates for evaluation to the input database. Guiding the patient or caregiver involves evaluating the input in real time to detect deficiencies in the entry of that data. Such a deficiency can include a skipped question, an entry containing letters or other characters when a number is the appropriate response, and the like. Guidance can take any suitable form. In some instances, an on-screen indication appears, highlighting the skipped question or the erroneous entry, for example. In further instances, a character such as a dog can appear to lend a friendly face to the guidance. Audible and/or visible guidance can be used, such as, for example, words explaining the guidance, a friendly bark if a dog character appears, a beep or jingle, an animation sequence of a dog wagging or chasing its tail, panting, or barking, or combinations thereof.

The communication engine can be configured to warn the patient and/or the caregiver when the change for the worse in any of the symptoms, the assessments, the social determinants of health, or the observables greater than one or more thresholds over time has been detected by the correlation engine. This warning can be delivered in any suitable manner. It can occur when the patient or caregiver enters the information indicating the change, and/or it can occur at any suitable point once the system has detected the change. On-screen warnings and audible signals may be used while the patient or caregiver is logged into the system, and a text message, an email message, an automated phone call, or a combination thereof may also be employed. Smartphone numbers for receiving text messages and automated phone calls and email addresses can be recorded in a patient profile. Similarly, the communication engine can be configured to encourage the patient and/or the caregiver when the change for the better in any of the symptoms, the assessments, the social determinants of health, or the observables greater than one or more thresholds over time has been detected by the correlation engine. This encouragement can be delivered in any suitable manner, such as, for example, in the same ways warnings can be delivered.

The system further comprises a dashboard engine configured to graphically display the symptoms, the assessments, the social determinants of health, the substrates for evaluation, the observables, the triggers, and/or the boosts. The dashboard engine provides a graphical depiction suitable for the patient's or caregiver's chosen device using any suitable method such as those known in the art. Any suitable device can be used, such as, for example, a smart phone, a smart watch, a tablet computer, a laptop computer, a desktop computer, a game console with a monitor, a television, or the like.

Methods for managing brain injury or brain malfunction in a patient in need thereof also appear in various embodiments of the present invention. Many of those methods use a system as described herein to perform the functions elaborated above and herein. As stated before, processors representing “the processor” can appear in different devices across a network such as the Internet. Accordingly, steps of the process can be performed in various locations. As can be appreciated, subordinate processors can be tasked with performing specific steps by a command processor or processors as part of the methods of the present invention. Any suitable network may be used. The internet, a private local area network, or a hybrid network, wherein certain information can be sought while much information is protected, can be mentioned. Public networks and invitation-only networks such as provided by Google®, Microsoft, universities, hospitals, health insurance groups, private employers, and government agencies such as the Veterans' Administration and National Institutes of Health also can be mentioned.

A non-transitory computer program product, as used herein, comprises computer-readable instructions set forth in non-volatile memory that when compiled or run by a system, cause the system to execute those instructions and perform the steps of the present invention. Non-transitory computer program products include any suitable tangible media, now existing or later developed, that include computer-executable code for performing the various steps of the inventive methods. Any suitable non-transitory computer program products can be included, such as, for example, floppy disks, hard disks, servers, flash drives, solid state drives, even magnetic tape and punch cards, if one is so inclined. Tangible devices that enable data storage and retrieval from “the cloud” also may be mentioned, such as, for example, servers, storage devices, and networking components. Electrical, magnetic, electromagnetic, and optical data storage devices may be mentioned. Transitory signals in a wire or transmitted or broadcast wirelessly are not included. The devices upon which the methods are performed, and that can represent the systems described herein, are not limited. General-purpose computers, specific-purpose computers, smart phones, smart watches, tablet computers, laptop computers, desktop computers, game consoles with monitors, televisions, servers, terminal-accessed mainframe computers, and the like may be mentioned.

Further embodiments relate to methods for training a system for managing brain injury or brain malfunction in a patient in need thereof, one such method comprising providing the system with an initial dataset comprising one or more of initial symptoms, initial assessments, initial social determinants of health, initial substrates for evaluation, initial observables, and initial anomalies; and identifying for the system initial triggers comprising negative correlations between one or more of the initial symptoms, the initial assessments, the initial social determinants of health, the initial substrates for evaluation, the initial observables, and the initial anomalies, identifying for the system initial boosts comprising positive correlations between one or more of the initial symptoms, the initial assessments, the initial social determinants of health, the initial substrates for evaluation, the initial observables, and the initial anomalies, thereby training the system. Optionally, a system can be trained only for triggers, only for boosts, or both triggers and boosts. Any suitable systems can be trained, such as, for example, the systems described herein.

Certain instances of the present invention relate to a neural network trained to identify triggers in a patient suffering from brain injury or brain malfunction, comprising a pre-populated library populated with one or more of initial symptoms, initial assessments, initial social determinants of health, initial substrates for evaluation, initial observables, initial anomalies, initial triggers, and initial boosts. Further instances relate to a neural network trained to identify boosts in a patient suffering from brain injury or brain malfunction, comprising a pre-populated library populated with one or more of initial symptoms, initial assessments, initial social determinants of health, initial substrates for evaluation, initial observables, initial anomalies, initial triggers, and initial boosts. A neural network, as used herein, indicates a network of artificial neurons capable of information processing. The systems as described herein can comprise or access such a neural network as they iteratively search for triggers among the data relating to the patient's brain trauma.

Further instances relate to libraries for a system for managing brain injury or brain malfunction in a patient in need thereof, comprising one or more of symptoms, assessments, social determinants of health, substrates for evaluation, observables, and anomalies, optionally triggers, and optionally boosts. A library may relate to a single specific patient, or it may contain data for more than one or even many patients, of course, organized to distinguish one patient's data from the next patient's data. The systems as described herein can access or comprise such a library, useful as it would be to assist the system in managing a particular patient's recovery from brain trauma. A library can exist in any suitable form, such as for example, in a non-transitory computer storage medium. Any suitable non-transitory computer storage medium can be used. Suitable non-transitory computer storage media include any electronic, magnetic, electromagnetic, and optical data storage media, such as, for example, floppy disks, hard disks, servers, flash drives, solid state drives, even magnetic tape and punch cards. Specifically excluded are transitory forms such as signals in a wire or wirelessly transmitted.

Additional further embodiments comprise a system that can identify triggers. For example, certain instances provide a system trained to identify triggers in a patient recovering from brain injury or brain malfunction, wherein the system identifies the triggers from negative correlations between one or more symptoms, one or more assessments, one or more social determinants of health, one or more substrates for evaluation, one or more observables, one or more anomalies, or a combination thereof. Other embodiments comprise a system that can identify boosts. For example, a system trained to identify boosts in a patient identifies the boosts from positive correlations between one or more symptoms, one or more assessments, one or more social determinants of health, one or more substrates for evaluation, one or more observables, one or more anomalies, or a combination thereof. Any suitable models can be used to find the correlations that identify the triggers and the boosts. Random Forest Classifier, Neural Net, K-Nearest Neighbors, and Decision Tree Classifier may be mentioned.

Still further instances comprise a system that can predict symptoms for a patient that has entered certain data. Thus some embodiments relate to a system trained to predict one or more symptoms in a patient recovering from brain injury or brain malfunction, wherein the system predicts the one or more symptoms having been trained on an initial dataset comprising one or more of initial symptoms, initial assessments, initial social determinants of health, initial substrates for evaluation, initial observables, and initial anomalies. Optionally, the initial dataset includes initial triggers, initial boosts, or specifically excludes initial triggers and/or initial boosts, and the system learns initial triggers and/or initial boosts from the initial dataset according to any suitable model. Once trained, the system can then predict symptoms in a new patient based on the input by the patient or her caregiver. Any suitable models can be used. Random Forest Classifier, Neural Net, K-Nearest Neighbors, and Decision Tree Classifier may be mentioned.

### DETAILED DESCRIPTION OF THE DRAWINGS

Further embodiments of the present invention can be described by reference to the accompanying drawings.

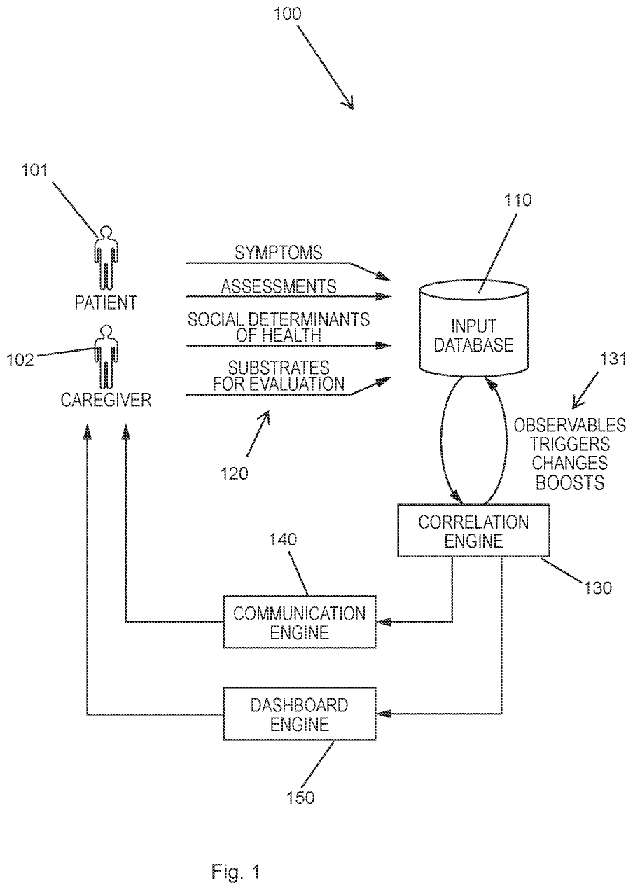

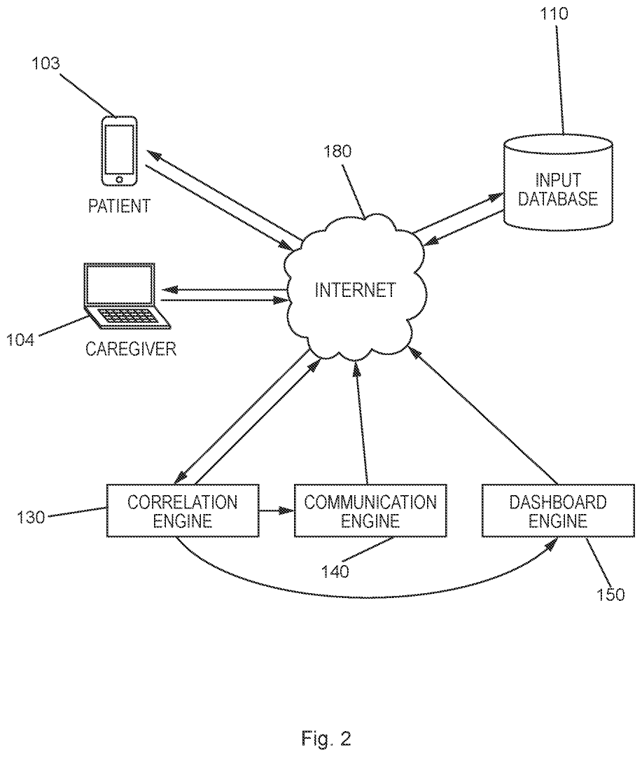

FIG. 1 schematically depicts one embodiment of the invention comprising system 100, while FIG. 2 schematically depicts components of system 100 in one possible configuration. System 100 for managing brain injury or brain malfunction in patient 101 in need thereof includes input database 110 configured to receive 120 symptoms, assessments, social determinants of health, and substrates for evaluation from patient 101 or her caregiver 102. Receiving 120 such data can occur at any suitable time, such as, for example, upon initial engagement of the patient, at a subsequent time as chosen by the patient 101 or caregiver 102, or when a medical professional prompts engagement of patient 101 or with assistance from caregiver 102. Receiving 120 such data can occur in any suitable manner. Patient 101 can use a smartphone 103, and/or caregiver 102 can use a laptop 104, for example. Input database 110 provides the data received 120 to correlation engine 130, which is configured to process the substrates for evaluation to obtain observables, correlate the symptoms, the assessments, and the social determinants of health from the input database, and the observables, to determine at least one trigger or at least one boost, detect a change in any of the symptoms, the assessments, the social determinants of health, or the observables greater than one or more thresholds over time, and indicate that one or more of additional symptoms, additional assessments, additional social determinants of health, and additional substrates for evaluation are needed. The observables, triggers, boosts, and changes can be stored 131 in the input database 110, or in another suitable memory storage structure. The correlation engine 130 works with communication engine 140, which is configured to reach out to the patient 101 or to the caregiver 102 to seek input of the symptoms, the assessments, the social determinants of health, the substrates for evaluation, the additional symptoms, the additional assessments, the additional social determinants of health, and the additional substrates for evaluation, guide the patient 101 or the caregiver 102 to input 120 the symptoms, the assessments, the social determinants of health, the substrates for evaluation, the additional symptoms, the additional assessments, the additional social determinants of health, and/or the additional substrates for evaluation to the input database 110, and warn the patient 101 or the caregiver 102 when the change in any of the symptoms, the assessments, the social determinants of health, or the observables greater than one or more thresholds over time has been detected by the correlation engine 130. The additional symptoms, the additional assessments, the additional social determinants of health, and the additional substrates for evaluation, once received 120 by the input database 110, are treated the same as the symptoms, the assessments, the social determinants of health, and the substrates for evaluation, respectively. Dashboard engine 150 is configured to graphically display the symptoms, the assessments, the social determinants of health, the substrates for evaluation, the observables, and/or the triggers for patient 101 and/or caregiver 102, for example on smartphone 103 and/or laptop 104. Any suitable network, such as for example internet 180, connects the several components of system 100 and smartphone 103 of patient 101 and laptop 104 of caregiver 104. It can be said that whoever provides or controls correlation engine 130 is responsible for the entire system 100. The system described in FIGS. 1 and 2 can be used to receive periodic or continuous data from patient 101 and caregiver 102, such as, for example, once a day, multiple times a day, or occasionally from time to time.

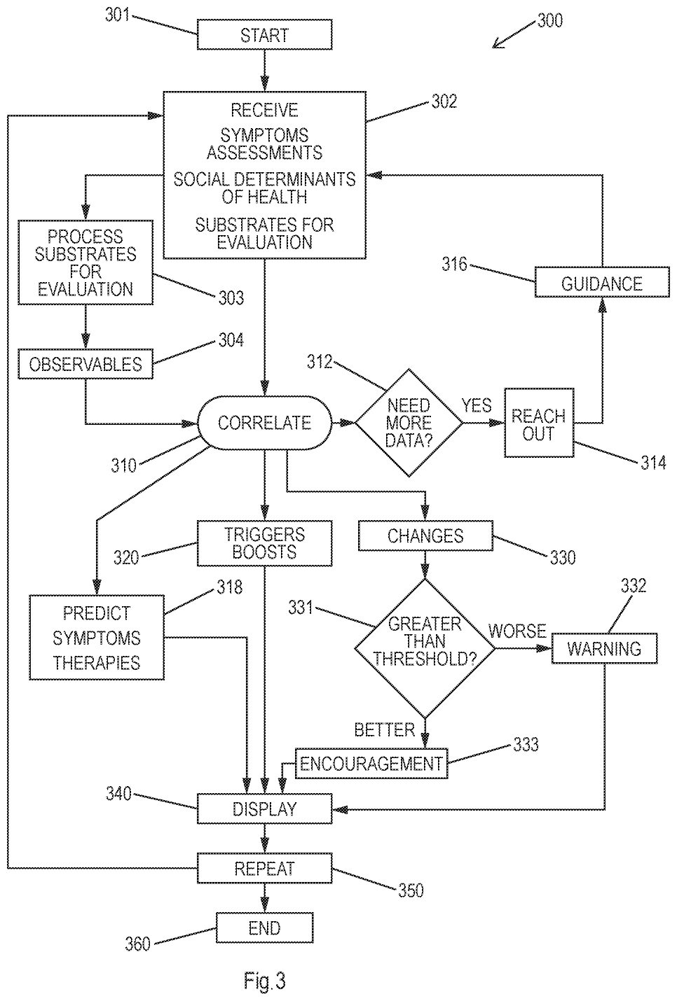

FIG. 3 provides a flowchart of method 300. Method 300 can be performed by a processor (not shown) that controls a system such as system 100 described in FIGS. 1 and 2. Method 300 starts 301 by receiving 302 from a patient or caregiver (for example, patient 101 and caregiver 102 of FIGS. 1 and 2), symptoms, assessments, social determinants of health, and substrates for evaluation. Substrates for evaluation are processed 303, using a processor, the substrates for evaluation to obtain observables 304. The processor determines whether additional data is needed 312, and reaches out 314 to the patient 101 or caregiver 102 to seek input of the symptoms, the assessments, the social determinants of health, the substrates for evaluation, the additional symptoms, the additional assessments, the additional social determinants of health, and the additional substrates for evaluation. While additional information is always helpful, for practical purposes, the answer to “need more data?” 312 is “no” if the correlations 310 have sufficient data to proceed. The processor may further provide guidance 316 patient 101 or caregiver 102 so that such additional data can be received 302. The processor correlates 310 the symptoms, the assessments, the social determinants of health, and the observables, to determine at least one trigger, at least one boost, or both 320. The processor, in this embodiment, also may predict 318 symptoms and successful therapies, for example, if there exists enough data on triggers and boosts 320. The processor also detects any changes 330 in any of the symptoms, the assessments, the social determinants of health, or the observables greater than one or more thresholds over time. If changes 330 for the worse in any of the symptoms, the assessments, the social determinants of health, or the observables are determined 331 to be greater than one or more thresholds over time as detected by the processor, the patient 101 or caregiver are provided warning 332 of the significant changes 330. If changes 330 for the better are determined 331 to be greater than one or more thresholds over time as detected by the processor, the patient 101 or caregiver 102 are provided encouragement 333. The processor can graphically display 340 the triggers and boosts 320, the changes 330, the warnings 332, and the encouragements 333 in addition to the symptoms, the assessments, the social determinants of health, the substrates for evaluation, and the observables 304. Steps of method 300 can be repeated 350. Method 300 can end 360, optionally to be repeated 350 at another time.

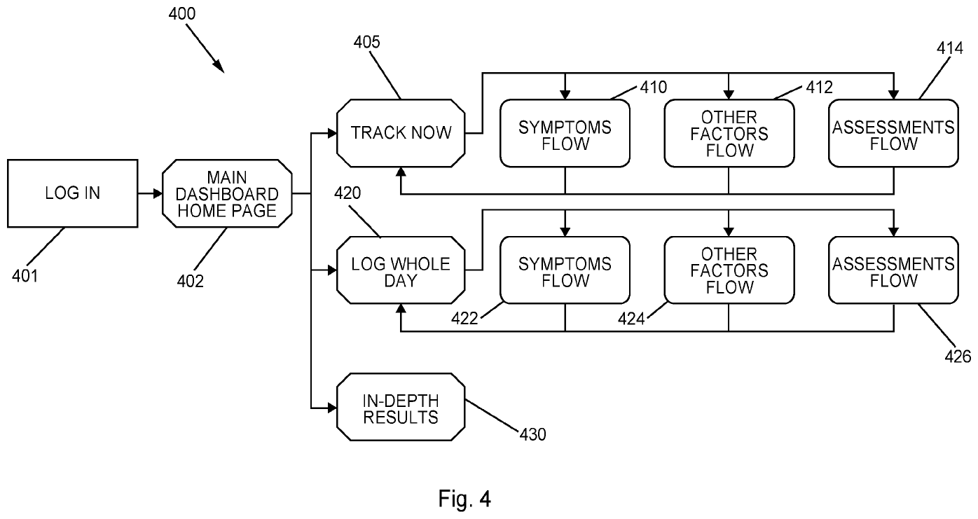

FIG. 4 provide a flowchart of method 400. Method 400 can form a portion of method 300, for example, and can be performed on system 100, for another example. Patient 101 or caregiver 102, for instance, can log in 401 to system 100 on smartphone 103 or laptop 104, and encounter a main dashboard home page 402, such as provided by dashboard engine 150 and informed by communication engine 140. Patient 101 is asked to decide what to track now 405. Symptoms are inputted by patient 101 in symptoms flow 410. As used herein, a “flow” is a series of prompts guiding a patient or a caregiver to provide data to the input database. Such data can be any suitable data, such as, for example, profile data, symptoms, assessments, social determinants of health, and substrates for evaluation. Social determinants of health and/or substrates for evaluation are inputted in response to the other factors flow 412. Assessments are inputted in response to the assessments flow 414. Alternatively, in method 400, patient 101 is asked whether to log the whole day 420. Symptoms flow 422 seeks input of symptoms, other factors flow 424 seeks social determinants of health and/or substrates for evaluation, and assessments flow 426 seeks input of assessments. From the main dashboard homepage, patient 101 has the opportunity to explore in-depth results 430, which can represent a graphical display of the symptoms, the assessments, the social determinants of health, the substrates for evaluation, the observables, the triggers, the changes in any of the symptoms, the assessments, the social determinants of health, or the observables, and the warnings when the changes are greater than one or more thresholds over time.

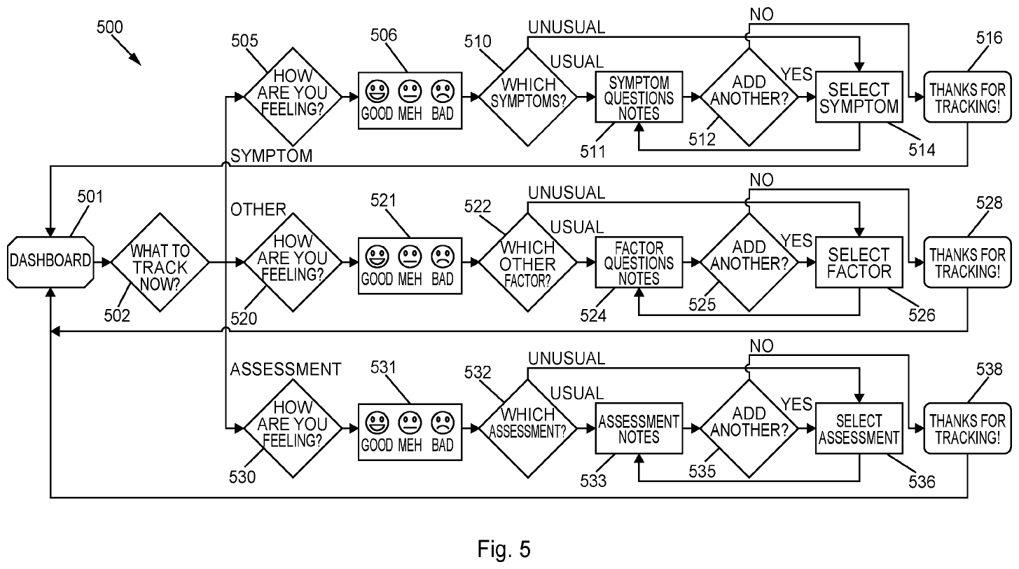

FIG. 5 provide a flowchart of method 500. Method 500 can form a portion of method 300, for example, and can be performed on system 100, for another example. From dashboard 501, a patient 101 operating smartphone 103, for example, is asked “what to track now?” 502. If patient 101 chooses symptoms, the question “how are you feeling?” 505 appears, along with a graphic 506 that allows the patient 101 to select “good,” “meh,” or “bad.” Once selected, specific symptoms can be explored from the question, “which symptom?” 510. Usual or previously-tracked symptoms are tracked by symptom question notes 511, and a list of such symptoms is expanded with “add another?” 512. A list of possible further symptoms is provided with a “select symptom” 514 opportunity, such as is provided by a dropdown menu. When finished, patient 101 encounters a “thanks for tracking!” message 516 and is returned to the dashboard 501. If, from the “what to track now?” 502, patient 101 wishes to track social determinants of health or provide substrates for evaluation, the question “how are you feeling?” 520 appears, along with a graphic 521 that allows the patient 101 to select “good,” “meh,” or “bad.” Once selected, specific social determinants of health or substrates for evaluation can be explored from the question, “which other factor?” 522. Usual or previously-tracked social determinants of health and substrates for evaluation are tracked by factor question notes 524, and a list of such factors is expanded with “add another?” 525. A list of possible further social determinants of health and substrates for evaluation is provided with a “select factor” 526 opportunity, such as is provided by a dropdown menu. When finished, patient 101 encounters a “thanks for tracking!” message 528 and is returned to the dashboard 501. If, from the “what to track now?” 502, patient 101 wishes to track assessments, the question “how are you feeling?” 530 appears, along with a graphic 531 that allows the patient 101 to select “good,” “meh,” or “bad.” Once selected, specific assessments can be explored from the question, “which assement?” 532. Usual or previously-tracked assessments are tracked by assment notes 533, and a list of such assemssments is expanded with “add another?” 535. A list of possible additional symptoms is provided with a “select assessment” 536 opportunity, such as is provided by a dropdown menu. When finished, patient 101 encounters a “thanks for tracking!” message 538 and is returned to the dashboard 501.

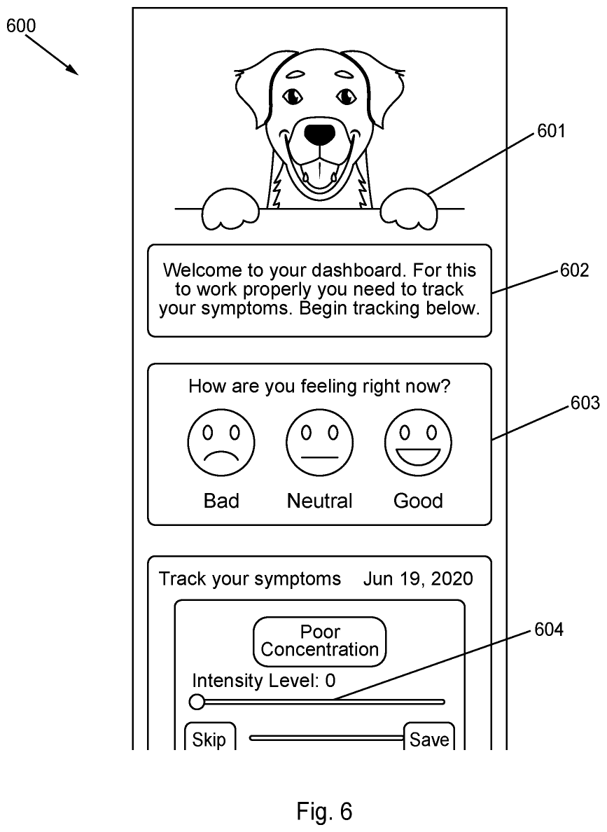

FIG. 6 shows a mockup screenshot of graphical user interface 600. Graphical user interface 600 can represent an execution of method 500 on smartphone 103, for example, and can represent a dashboard or a portion of a dashboard. Sallie the dog 601 can assist with the execution by offering a friendly face, and optionally sounds such as a friendly bark, panting, whining, or a combination thereof. Welcome message 602 provides guidance to track symptoms. Graphic 603 offers patient 101 to input a general feeling of “bad,” “neutral,” or “good,” and corresponds to graphic 506 in method 500. The patient is asked to track the symptom of “poor concentration,” and a slider bar 604 offers the opportunity to provide a numerical value for the intensity of the symptom. As used herein, the fact that the patient is suffering from poor concentration is described as a symptom, while the numerical value the patient assigns to the intensity of that symptom is described as an assessment.

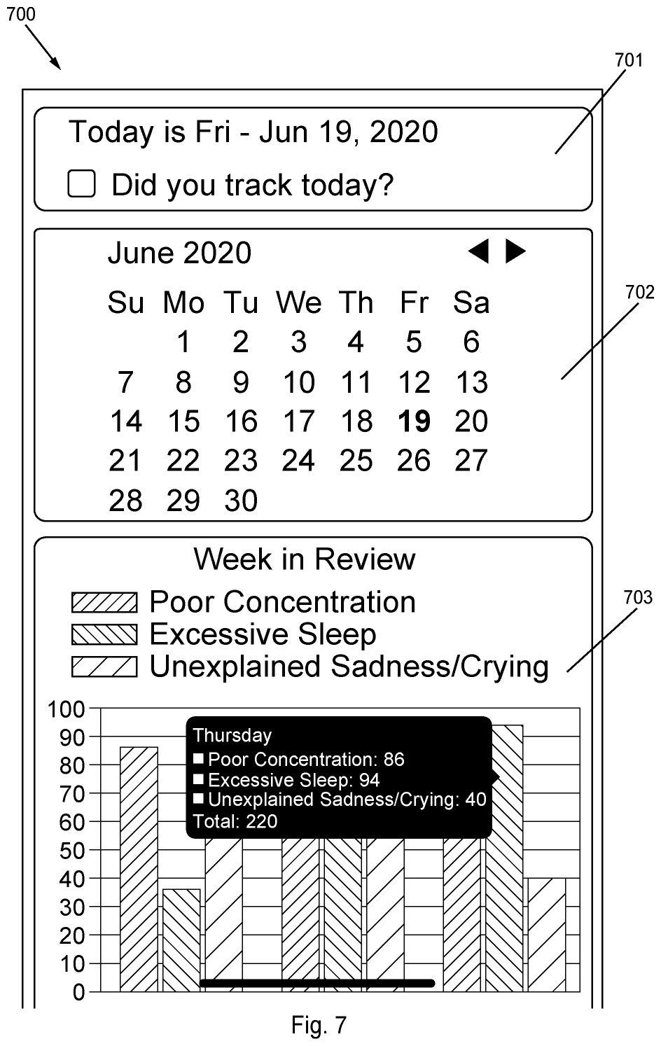

FIG. 7 shows a mockup screenshot of graphical user interface 700. Graphical user interface 700 can represent an execution of method 300 on smartphone 103, for example, and can represent a dashboard or a portion of a dashboard. Question 701 “did you track today?” could represent guidance to patient 101 to seek input of symptoms, assessments, social determinants of health, substrates for evaluation, additional symptoms, additional assessments, additional social determinants of health, and/or additional substrates for evaluation. Calendar 702 allows the patient 101 to select a date for tracking. Graphic 103 shows how assessments for three symptoms, “poor concentration,” “excessive sleep,” and “unexplained sadness/crying” have changed over a week. Graphic 703 also shows a summation of the assessments for those three symptoms for a single day, Thursday. Such a summation can show general trends in combined assessments, even if individual assessments vary dramatically.

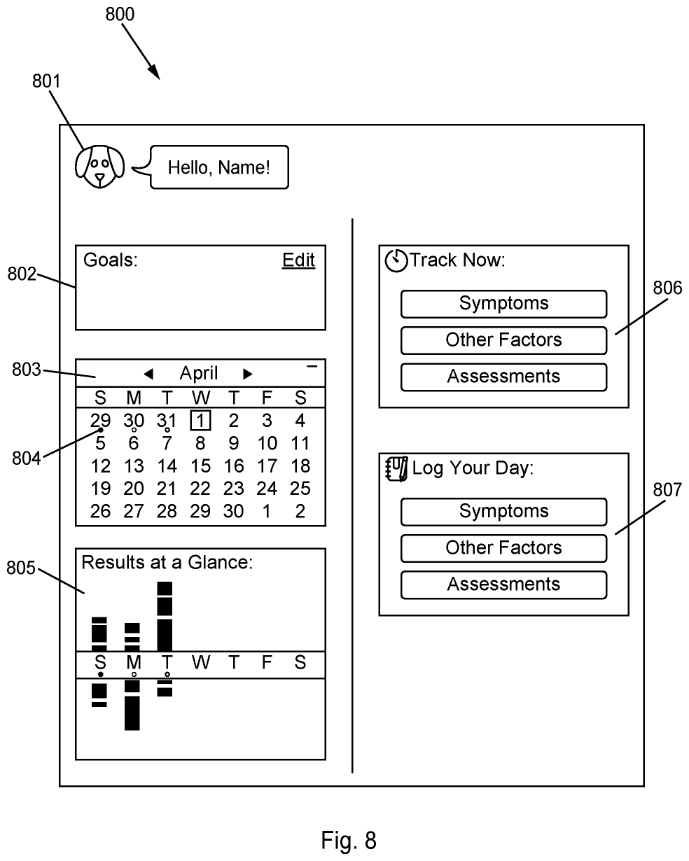

FIG. 8 shows a mockup screenshot of graphical user interface 800. Graphical user interface can represent an execution of method 300 on laptop 104, for example, and can represent a dashboard or a portion of a dashboard. Guidance can be facilitated with a graphic 801 of a dog. Goals 802, appearing in some further embodiments of the present invention, can reflect any suitable aspirations for the patient. In some cases, the patient can generate one or more goals, such as, for example, “Determine what causes my headaches.” In other cases, the correlation engine 130 (see FIGS. 1 and 2) can use the goals 802 dialogue to indicate that one or more additional symptoms, additional assessments, additional social determinants of health, and additional substrates for evaluation are needed, such as, for example, “Track more factors [e.g., social determinants of health] so the cause of headaches can be revealed.” Calendar 803 can be made interactive, so patient 101 or caregiver 102 can select any particular date to input data or look at the data input for any particular date. Color indicator 804 can be used to indicate the overall relative health of the patient, for example, by color-coding the sum of three assessments as shown in graphic 703 (see FIG. 7). Bar graph 805 shows results at a glance for five inputs for three days. Graphic 806 invites the viewer (patient 101 or caregiver 102) to track symptoms, other factors (e.g., social determinants of health and substrates for evaluation), and assessments especially if symptoms are flaring. Graphic 807 makes an invitation similar to graphic 806 but is geared toward a routine daily entry whether symptoms flair or not.

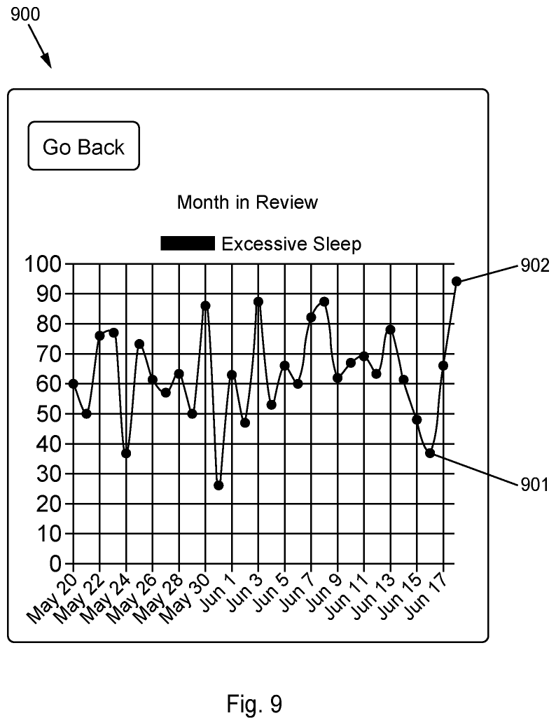

FIG. 9 depicts mockup graph 900. If patient 101 or caregiver 102 wishes to know how a particular symptom has modulated over time, the system can display, for example, the data of assessments of the intensity of that symptom on smartphone 103 or laptop 104. After declining for three days to an intensity below 40 at point 901 (June 16), the intensity shoots up to above 90 at point 902. In some cases, because the change exceeds say, a 30% threshold in each of two days, the system could warn the patient or the caregiver of the change and encourage them to seek help. Also, the system could identify June 17 as a landmark and search for triggers that caused or contributed to the excessive sleep. More than one threshold could be monitored to cause the system to perform different actions. For example, a 10% change could elicit a comment on the dashboard, while a 30% change could inspire a warning, encouragement, and/or a request for more information to help identify the cause of the dramatic change.

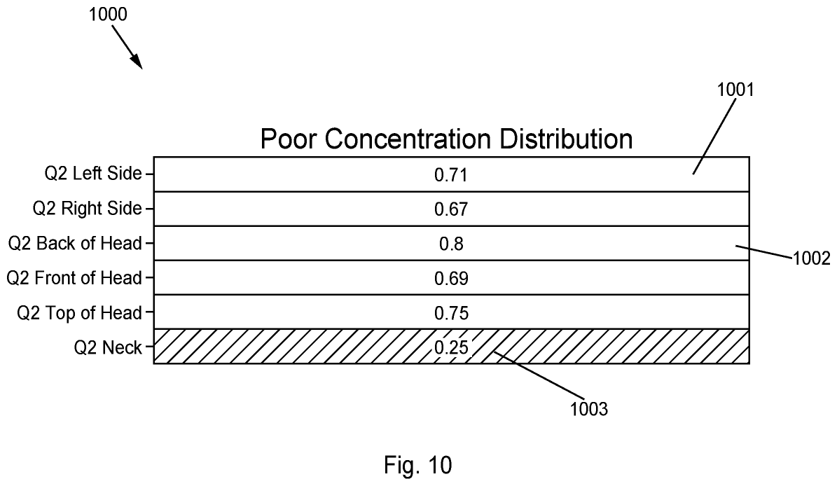

FIG. 10 depicts table 1000. Table 1000 shows, for a collection of patients recovering from brain injury due to a blow to the head or neck, the occurrence of poor concentration symptom for the patient. For patients who suffered a blow to the left side of the head 1001, the occurrence of the symptom is 0.71. For patients who suffered a blow to the back of the head 1002, the occurrence of the symptom is 0.8. It can be determined, in further embodiments of the present invention, that blows to the left side or back of the head may be a trigger for poor concentration. However, with an occurrence of just 0.25, blows to the neck 1003 are less strongly associated with the symptom of poor concentration. This table 1000 thus relates to embodiments of the present invention that involve training the system to look for triggers based on the data of many patients.

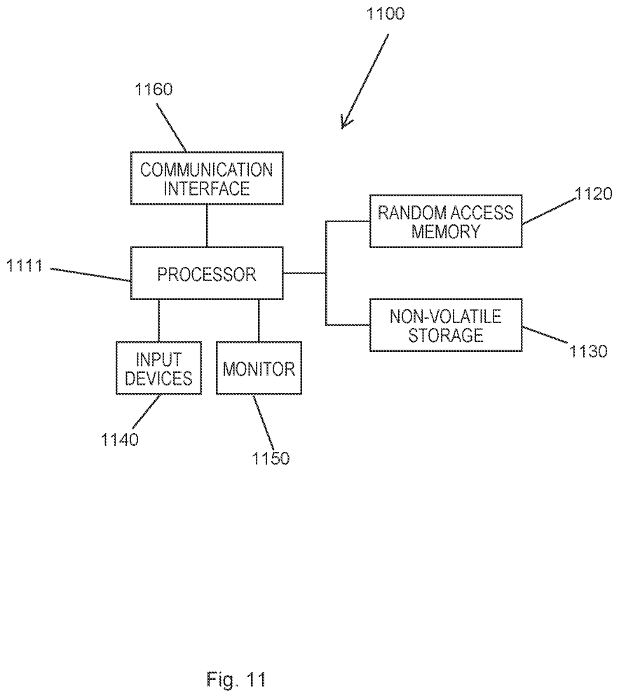

FIG. 11 schematically depicts hardware system 1100. Systems useful in the present invention, such as system 1100, can be any suitable computer system. Desktop, laptop, tablet, smartphone, smartwatch, server, and mainframe computers may be mentioned. With reference to FIG. 1, all or a portion of system 100 can reside on hardware system 1100, comprising processor 1111 coupled to random access memory 1120 and non-volatile memory 1130, such as a hard disk drive, floppy disk, CD-ROMs, DVDs, flash drives, and the like. Such random-access memory 1120 and non-volatile memory 1130 can provide non-volatile storage for program and data files, and can be configured to receive, for example, the symptoms, assessments, social determinants of health, and substrates for evaluation. Such random-access memory 1120 and non-volatile memory 1130 also can provide a physical location for the non-transitory computer programs operable upon execution by the processor for performing inventive processes according to the present invention. Such computer programs can be loaded into random access memory 1120 when the processor is ready to run those programs; random access memory 1120 also can temporarily store any suitable data such as the symptoms, assessments, social determinants of health, and substrates for evaluation for correlation or other processing by processor 1111. Any suitable processor 1111 can be used. For example, processor 1111 may comprise multiple processors, operating in concert to facilitate or quicken necessary operations. Input devices 1140 can include any suitable input devices, such as, for example, keyboard, mouse, microphone, camera, touchscreen, and combinations thereof. Monitor 1150 allows a person using hardware system 1100 to observe the workings of system 1100, and in some cases, allows for the display of a dashboard displaying, for example, changes in symptoms, assessments, social determinants of health, and substrates for evaluation, observables, and triggers. Hardware system 1100 also provides communication interface 1160 to allow processor 1111 to reach out to the patient or the caregiver to seek input of the symptoms, assessments, social determinants of health, and the substrates for evaluation, and otherwise perform the steps of the inventive method.

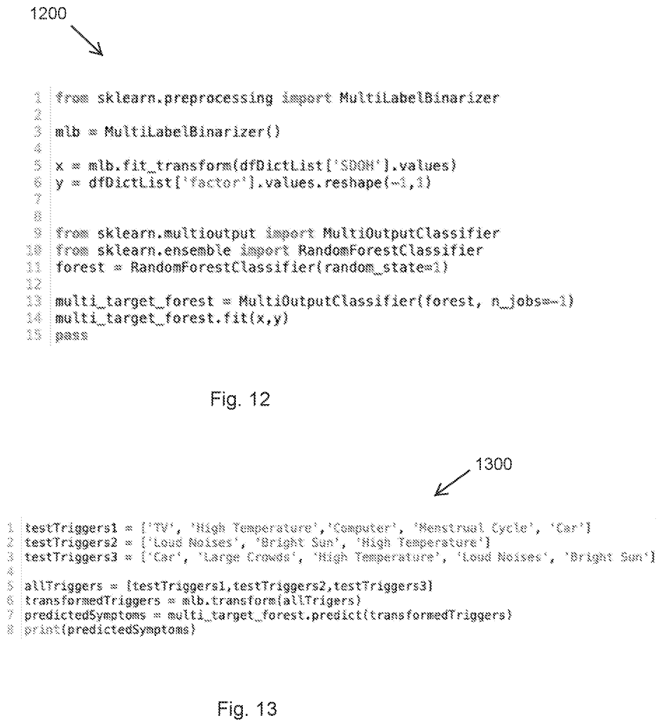

FIG. 12 shows code 1200 suitable for training a system using a Random Forest Classifier. The code causes the system to search for patterns between, for example, initial symptoms and initial social determinants of health for a number of patients. When those patterns are identified, the system potentially can predict symptoms given social determinants of health or can identify triggers among social determinants of health for a specific patient using the trained system.

FIG. 13 shows code 1300 suitable for predicting symptoms for patients having entered social determinants of health. Suppose the system has been trained as shown in FIG. 12. If Patient 1 uses the trained system and inputs “TV,” “High Temperature,” “Computer,” “Menstrual Cycle,” and “Car” as social determinants of health, code 1300 causes the system to predict symptoms of “Headaches and/or Migraines.” If Patient 2 uses the trained system and inputs “Loud Noises,” “Bright Sun,” and “High Temperature” as social determinants of health, code 1300 causes the system to predict symptoms of “Loss of Balance/Dizziness.” If Patient 3 inputs “Car,” “Large Crowds,” “High Temperature,” “Loud Noises,” and “Bright Sun,” code 1300 causes the system to predict “Fatigue” for Patient 3.

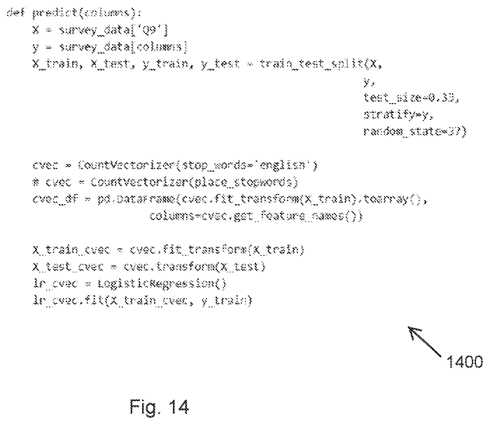

FIG. 14 shows code 1400 suitable for predicting symptoms for patients having entered a written narrative as a substrate for evaluation. Using actual patient data on thirty-three symptoms, a trained system according to the present invention was able to predict five symptoms with 87.5% accuracy, twelve symptoms with a 75% accuracy, ten symptoms with a 62.5% accuracy, and six symptoms with a 50% accuracy.

### EMBODIMENTS

Further aspects of the invention can be understood from the following embodiments.

Embodiment 1. A system for managing brain injury or brain malfunction in a patient in need thereof, comprising:

an input database configured to receive

symptoms,

assessments,

social determinants of health, and

substrates for evaluation;

a correlation engine configured to
(a) process the substrates for evaluation from the input database to obtain observables;
(b) correlate the symptoms, the assessments, and the social determinants of health from the input database, and the observables, to determine at least one trigger, at least one boost, or both;
(c) detect a change for the worse or a change for the better in any of the symptoms, the assessments, the social determinants of health, or the observables greater than one or more thresholds over time; and
(d) indicate that one or more of additional symptoms, additional assessments, additional social determinants of health, and additional substrates for evaluation are needed;
a communication engine configured to
(i) reach out to the patient or to a caregiver thereof to seek input of the symptoms, the assessments, the social determinants of health, the substrates for evaluation, the additional symptoms, the additional assessments, the additional social determinants of health, and the additional substrates for evaluation;
(ii) guide the patient or the caregiver to input the symptoms, the assessments, the social determinants of health, the substrates for evaluation, the additional symptoms, the additional assessments, the additional social determinants of health, and/or the additional substrates for evaluation to the input database, wherein the additional symptoms, the additional assessments, the additional social determinants of health, and the additional substrates for evaluation, once inputted into the input database, are treated the same as the symptoms, the assessments, the social determinants of health, and the substrates for evaluation, respectively; and
(iii) warn the patient or the caregiver when the change for the worse in any of the symptoms, the assessments, the social determinants of health, or the observables greater than one or more thresholds over time has been detected by the correlation engine;
(iv) encourage the patient or the caregiver when the change for the better in any of the symptoms, the assessments, the social determinants of health, or the observables for the better greater than one or more thresholds over time has been detected by the correlation engine; and
a dashboard engine configured to graphically display the symptoms, the assessments, the social determinants of health, the substrates for evaluation, the observables, the at least one trigger, and/or the at least one boost,
thereby managing the brain injury or brain malfunction in the patient.

Embodiment 2. The system of embodiment 1, wherein the symptoms comprise one or more of headache, nausea, dizziness, fatigue, oversleeping, difficulty sleeping, loss of vocabulary, slurred speech, sensitivity to light, sensitivity to sound, forgetfulness, difficulty concentrating, changes in mood, anxiousness, depression, loss of motivation, loss of appetite, and seizures.

Embodiment 3. The system of any one of the preceding embodiments, wherein the assessments comprise the patient or the caregiver assigning a numerical value to one or more of mood, quality of sleep, quantity of sleep, level of appetite, ability to concentrate, mental function, emotional function, sociability, physical activity, and quality of life.

Embodiment 4. The system of any one of the preceding embodiments, wherein the social determinants of health comprise one or more of weather, outside temperature, inside temperature, transportation, physical activity, social activity, financial well-being, race, color, religion, and gender.

Embodiment 5. The system of any one of the preceding embodiments, wherein the substrates for evaluation comprise one or more of an image of the patient's eyes including eyelids, an image of the patient's face, a narrative written or spoken by the patient, and a brain game played by the patient.

Embodiment 6. The system of any one of the preceding embodiments, wherein the observables comprise:

(a1) a grade of a narrative written or spoken by the patient;
(a2) an identification of an anomaly;
(a3) a brain game outcome; or
a combination thereof.

Embodiment 7. The system of embodiment 6, wherein the anomaly comprises asymmetrical eyelids of the patient.

Embodiment 8. The system of any one of the preceding embodiments, wherein the at least one trigger represents a negative relationship between at least one of the social determinants of health and at least one of the symptoms, at least one of the assessments, at least one of the observables, or a combination thereof.

Embodiment 9. The system of any one of the preceding embodiments, wherein the at least one boost represents a positive relationship between at least one of the social determinants of health and at least one of the symptoms, at least one of the assessments, at least one of the observables, or a combination thereof.

Embodiment 10. The system of any one of the preceding embodiments, wherein the one or more thresholds over time represents a change of greater than about 5%, greater than about 10%, greater than about 20%, greater than greater than about 25%, greater than about 30%, greater than about 40%, greater than about 50%, greater than about 75%, greater than about 90%, or greater than about 100%.

Embodiment 11. The system of any one of the preceding embodiments, wherein one or more of the additional symptoms, the additional assessments, the additional social determinants of health, and/or the additional substrates for evaluation are indicated as needed by a lack of receipt of the symptoms, the assessments, the social determinants of health, and/or the substrates for evaluation, respectively, for a given period.

Embodiment 12. The system of any one of the preceding embodiments, wherein the communication engine configured to (i) reach out to the patient or the caregiver comprises the system sending to the patient and/or the caregiver at least one electronic communication chosen from a text message, an email message, an automated phone call, or a combination thereof.

Embodiment 13. A method for managing brain injury or brain malfunction in a patient in need thereof, comprising:

receiving in memory

symptoms,

assessments,

social determinants of health, and

substrates for evaluation;

processing, using a processor, the substrates for evaluation to obtain observables;
correlating, using the processor, the symptoms, the assessments, the social determinants of health, and the observables, to determine at least one trigger, at least one boost, or both;
detecting, using the processor, a change for the worse or a change for the better in any of the symptoms, the assessments, the social determinants of health, or the observables greater than one or more thresholds over time;
indicating, using the processor, that one or more of additional symptoms, additional assessments, additional social determinants of health, and additional substrates for evaluation are needed;
reaching out, using the processor, to the patient or to a caregiver thereof to seek input of the symptoms, the assessments, the social determinants of health, the substrates for evaluation, the additional symptoms, the additional assessments, the additional social determinants of health, and the additional substrates for evaluation;
guiding, using the processor, the patient or the caregiver to input the symptoms, the assessments, the social determinants of health, the substrates for evaluation, the additional symptoms, the additional assessments, the additional social determinants of health, and/or the additional substrates for evaluation into memory,
wherein the additional symptoms, the additional assessments, the additional social determinants of health, and the additional substrates for evaluation, once received by memory, are treated the same as the symptoms, the assessments, the social determinants of health, and the substrates for evaluation, respectively;
warning, using the processor, the patient or the caregiver when the change for the worse in any of the symptoms, the assessments, the social determinants of health, or the observables greater than one or more thresholds over time has been detected by the processor;
encouraging, using the processor, the patient or the caregiver when the change for the better in any of the symptoms, the assessments, the social determinants of health, or the observables greater than one or more thresholds over time has been detected by the processor;
graphically displaying, using the processor, the symptoms, the assessments, the social determinants of health, the substrates for evaluation, the observables, the at least one trigger, and/or the at least one boost,
thereby managing the brain injury or brain malfunction in the patient.

Embodiment 14. The method of embodiment 13, wherein the symptoms comprise one or more of headache, nausea, dizziness, fatigue, oversleeping, difficulty sleeping, loss of vocabulary, slurred speech, sensitivity to light, sensitivity to sound, forgetfulness, difficulty concentrating, changes in mood, anxiousness, depression, loss of motivation, loss of appetite, and seizures.

Embodiment 15. The system of any one of embodiments 13-14, wherein the assessments comprise assigning, using the processor, a numerical value to one or more of mood, quality of sleep, quantity of sleep, level of appetite, ability to concentrate, mental function, emotional function, sociability, physical activity, and quality of life.

Embodiment 16. The process of any one of embodiments 13-15, wherein the social determinants of health comprise one or more of weather, outside temperature, inside temperature, transportation, physical activity, social activity, financial well-being, race, color, religion, and gender.

Embodiment 17. The process of any one of embodiments 13-16, wherein the substrates for evaluation comprise one or more of an image of the patient's eyes including eyelids, an image of the patient's face, a narrative written or spoken by the patient, and a brain game played by the patient.

Embodiment 18. The process of any one of embodiments 13-17, wherein the observables comprise:

(a1) a grade of a narrative written or spoken by the patient;
(a2) an identification of an anomaly;
(a3) a brain game outcome; or
a combination thereof.

Embodiment 19. The process of embodiment 18, wherein the anomaly comprises asymmetrical eyelids of the patient.

Embodiment 20. The process of any one of embodiments 13-19, wherein the at least one trigger represents a negative relationship between at least one of the social determinants of health and at least one of the symptoms, at least one of the assessments, at least one of the observables, or a combination thereof.

Embodiment 21. The process of any one of embodiments 13-20, wherein the at least one boost represents a positive relationship between at least one of the social determinants of health and at least one of the symptoms, at least one of the assessments, at least one of the observables, or a combination thereof.

Embodiment 22. The process of any one of embodiments 13-21, wherein the one or more thresholds over time represents a change of greater than about 5%, greater than about 10%, greater than about 20%, greater than greater than about 25%, greater than about 30%, greater than about 40%, greater than about 50%, greater than about 75%, greater than about 90%, or greater than about 100%.

Embodiment 23. The process of any one of embodiments 13-22, wherein one or more of the additional symptoms, the additional assessments, the additional social determinants of health, and/or the additional substrates for evaluation are indicated as needed by a lack of the receiving in memory of the symptoms, the assessments, the social determinants of health, and/or the substrates for evaluation, respectively, for a given period.

Embodiment 24. The process of any one of embodiments 13-23, wherein the reaching out to the patient or the caregiver comprises sending, using the processor, to the patient and/or the caregiver at least one electronic communication chosen from a text message, an email message, an automated phone call, or a combination thereof.

Embodiment 25. The process of any one of embodiments 13-24, wherein the reaching out to the patient or the caregiver comprises sending, using the processor, a prompt to a medical care professional to call, send an email, or otherwise message the patient or the caregiver.

Embodiment 26. The process of any one of embodiments 13-25, wherein the process is performed on the system of any one of embodiments 1-12.

Embodiment 27. A non-transitory computer program product comprising a program code that, upon execution by a processor, is configured to perform a method for managing brain injury or brain malfunction in a patient in need thereof, comprising:

receiving in memory

symptoms,

assessments,

social determinants of health, and

substrates for evaluation;

processing, using a processor, the substrates for evaluation to obtain observables; correlating, using the processor, the symptoms, the assessments, the social determinants of health, and the observables, to determine at least one trigger, at least one boost, or both;
detecting, using the processor, a change for the worse or a change for the better in any of the symptoms, the assessments, the social determinants of health, or the observables greater than one or more thresholds over time;
indicating, using the processor, that one or more of additional symptoms, additional assessments, additional social determinants of health, and additional substrates for evaluation are needed;
reaching out, using the processor, to the patient or to a caregiver thereof to seek input of the symptoms, the assessments, the social determinants of health, the substrates for evaluation, the additional symptoms, the additional assessments, the additional social determinants of health, and the additional substrates for evaluation;
guiding, using the processor, the patient or the caregiver to input the symptoms, the assessments, the social determinants of health, the substrates for evaluation, the additional symptoms, the additional assessments, the additional social determinants of health, and/or the additional substrates for evaluation into memory,
wherein the additional symptoms, the additional assessments, the additional social determinants of health, and the additional substrates for evaluation, once received by memory, are treated the same as the symptoms, the assessments, the social determinants of health, and the substrates for evaluation, respectively;
warning, using the processor, the patient or the caregiver when the change for the worse in any of the symptoms, the assessments, the social determinants of health, or the observables greater than one or more thresholds over time has been detected by the processor;
encouraging, using the processor, the patient or the caregiver when the change for the better in any of the symptoms, the assessments, the social determinants of health, or the observables greater than one or more thresholds over time has been detected by the processor;
graphically displaying, using the processor, the symptoms, the assessments, the social determinants of health, the substrates for evaluation, the observables, the at least one trigger, and/or the at least one boost,
thereby managing the brain injury or brain malfunction in the patient.

Embodiment 28. The non-transitory computer program product of embodiment 27, wherein the method is the process of any one of embodiments 13-26.

Embodiment 29. A method of training a system for managing brain injury or brain malfunction in a patient in need thereof, comprising:

providing the system with an initial dataset comprising one or more of initial symptoms, initial assessments, initial social determinants of health, initial substrates for evaluation, initial observables, and initial anomalies; and
identifying for the system initial triggers comprising negative correlations between one or more of the initial symptoms, the initial assessments, the initial social determinants of health, the initial substrates for evaluation, the initial observables, and the initial anomalies,
identifying for the system initial boosts comprising positive correlations between one or more of the initial symptoms, the initial assessments, the initial social determinants of health, the initial substrates for evaluation, the initial observables, and the initial anomalies,
thereby training the system.

Embodiment 30. The method of embodiment 29, wherein the system is any one of the systems of any one of embodiments 1-12.

Embodiment 31. A neural network trained to identify triggers in a patient suffering from brain injury or brain malfunction, comprising a pre-populated library populated with one or more of initial symptoms, initial assessments, initial social determinants of health, initial substrates for evaluation, initial observables, initial anomalies, initial triggers, and initial boosts.

Embodiment 32. A neural network trained to identify boosts in a patient suffering from brain injury or brain malfunction, comprising a pre-populated library populated with one or more of initial symptoms, initial assessments, initial social determinants of health, initial substrates for evaluation, initial observables, initial anomalies, initial triggers, and initial boosts.

Embodiment 33. The system of any one of embodiments 1-12, further comprising the neural network of any one of embodiments 31-32.

Embodiment 34. A library for a system for managing brain injury or brain malfunction in a patient in need thereof, comprising one or more of symptoms, assessments, social determinants of health, substrates for evaluation, observables, and anomalies, optionally triggers, and optionally boosts.

Embodiment 35. A non-transitory computer storage medium comprising the library of embodiment 34.

Embodiment 36. The system of any one of embodiments 1-12 and 33, further comprising the library of any one of embodiments 34-35.

Embodiment 37. A system trained to identify triggers in a patient recovering from brain injury or brain malfunction, wherein the system identifies the triggers from negative correlations between one or more symptoms, one or more assessments, one or more social determinants of health, one or more substrates for evaluation, one or more observables, one or more anomalies, or a combination thereof.

Embodiment 38. A system trained to identify boosts in a patient recovering from brain injury or brain malfunction, wherein the system identifies the boosts from positive correlations between one or more symptoms, one or more assessments, one or more social determinants of health, one or more substrates for evaluation, one or more observables, one or more anomalies, or a combination thereof.

Embodiment 39. A system trained to predict one or more symptoms in a patient recovering from brain injury or brain malfunction, wherein the system predicts the one or more symptoms having been trained on an initial dataset comprising one or more of initial symptoms, initial assessments, initial social determinants of health, initial substrates for evaluation, initial observables, and initial anomalies.

Embodiment 40. The system of embodiment 39, wherein the initial data set comprises one or more initial triggers.

Embodiment 41. The system of embodiment 39, wherein the initial dataset does not include initial triggers.

Embodiment 42. The system of any one of embodiments 39-41, wherein the initial data set comprises one or more initial boosts.

Embodiment 43. The system of any one of embodiments 39-41, wherein the initial dataset does not include initial boosts.

Embodiment 44. The system of any one of embodiments 37-43, wherein the system is the system of any one of embodiments 1-12, 33, and 36.

Embodiment 45. A method of managing brain recovery in a patient in need thereof, comprising:

identifying one or more triggers inhibiting brain recovery;
communicating to the patient to reduce, avoid, or otherwise minimize exposure to the one or more triggers;
identifying one or more boosts enhancing brain recovery;
communicating to the patient to increase, pursue, or otherwise maximize exposure to the one or more boosts,
thereby managing brain recovery in the patient.

Embodiment 46. A method of managing brain function improvement in

a patient in need thereof, comprising:
Identifying one or more boosts enhancing brain function;
communicating to the patient to increase, pursue, or otherwise maximize exposure to the one or more boosts,
thereby managing brain function improvement in the patient.

As previously stated, detailed embodiments of the present invention are disclosed herein; however, it is to be understood that the disclosed embodiments are merely exemplary of the invention that may be embodied in various forms. It will be appreciated that many modifications and other variations stand within the intended scope of this invention as claimed below. Furthermore, the foregoing description of various embodiments does not necessarily imply exclusion. For example, “some” embodiments may include all or part of “other” and “further” embodiments within the scope of this invention. In addition, “a” does not mean “one and only one;” “a” can mean “one and more than one.”

1. A system for managing brain injury or brain malfunction in a patient in need thereof, comprising:
an input database configured to receive
symptoms,
assessments,
social determinants of health, and
substrates for evaluation;
a correlation engine configured to
(a) process the substrates for evaluation from the input database to obtain observables;
(b) correlate the symptoms, the assessments, and the social determinants of health from the input database, and the observables, to determine at least one trigger, at least one boost, or both;
(c) detect a change for the worse or a change for the better in any of the symptoms, the assessments, the social determinants of health, or the observables greater than one or more thresholds over time; and
(d) indicate that one or more of additional symptoms, additional assessments, additional social determinants of health, and additional substrates for evaluation are needed;
a communication engine configured to
(i) reach out to the patient or to a caregiver thereof to seek input of the symptoms, the assessments, the social determinants of health, the substrates for evaluation, the additional symptoms, the additional assessments, the additional social determinants of health, and the additional substrates for evaluation;
(ii) guide the patient or the caregiver to input the symptoms, the assessments, the social determinants of health, the substrates for evaluation, the additional symptoms, the additional assessments, the additional social determinants of health, and/or the additional substrates for evaluation to the input database,
wherein the additional symptoms, the additional assessments, the additional social determinants of health, and the additional substrates for evaluation, once inputted into the input database, are treated the same as the symptoms, the assessments, the social determinants of health, and the substrates for evaluation, respectively; and
(iii) warn the patient or the caregiver when the change for the worse in any of the symptoms, the assessments, the social determinants of health, or the observables greater than one or more thresholds over time has been detected by the correlation engine;
(iv) encourage the patient or the caregiver when the change for the better in any of the symptoms, the assessments, the social determinants of health, or the observables for the better greater than one or more thresholds over time has been detected by the correlation engine; and
a dashboard engine configured to graphically display the symptoms, the assessments, the social determinants of health, the substrates for evaluation, the observables, the at least one trigger, and/or the at least one boost,
thereby managing the brain injury or brain malfunction in the patient.

2. The system of claim 1, wherein the symptoms comprise one or more of headache, nausea, dizziness, fatigue, oversleeping, difficulty sleeping, loss of vocabulary, slurred speech, sensitivity to light, sensitivity to sound, forgetfulness, difficulty concentrating, changes in mood, anxiousness, depression, loss of motivation, loss of appetite, and seizures.

3. The system of claim 1, wherein the assessments comprise the patient or the caregiver assigning a numerical value to one or more of mood, quality of sleep, quantity of sleep, level of appetite, ability to concentrate, mental function, emotional function, sociability, physical activity, and quality of life.

4. The system of claim 1, wherein the social determinants of health comprise one or more of weather, outside temperature, inside temperature, transportation, physical activity, social activity, financial well-being, race, color, religion, and gender.

5. The system of claim 1, wherein the substrates for evaluation comprise one or more of an image of the patient's eyes including eyelids, an image of the patient's face, a narrative written or spoken by the patient, and a brain game played by the patient.

6. The system of claim 1, wherein the observables comprise:
(a1) a grade of a narrative written or spoken by the patient;
(a2) an identification of an anomaly;
(a3) a brain game outcome; or
a combination thereof.

7. The system of claim 6, wherein the anomaly comprises asymmetrical eyelids of the patient.

8. The system of claim 1, wherein the at least one trigger represents a negative relationship between at least one of the social determinants of health and at least one of the symptoms, at least one of the assessments, at least one of the observables, or a combination thereof.

9. The system of claim 1, wherein the at least one boost represents a positive relationship between at least one of the social determinants of health and at least one of the symptoms, at least one of the assessments, at least one of the observables, or a combination thereof.

10. The system of claim 1, wherein the one or more thresholds over time represents a change of greater than about 10%.

11. The system of claim 1, wherein one or more of the additional symptoms, the additional assessments, the additional social determinants of health, and/or the additional substrates for evaluation are indicated as needed by a lack of receipt of the symptoms, the assessments, the social determinants of health, and/or the substrates for evaluation, respectively, for a given period.

12. The system of claim 1, wherein the communication engine configured to (i) reach out to the patient or the caregiver comprises the system sending to the patient and/or the caregiver at least one electronic communication chosen from a text message, an email message, an automated phone call, or a combination thereof.

13-38. (canceled)

39. The system of claim 1, wherein the system is trained to predict one or more symptoms in a patient recovering from brain injury or brain malfunction, the system having been trained on an initial dataset comprising one or more of initial symptoms, initial assessments, initial social determinants of health, initial substrates for evaluation, initial observables, and initial anomalies.

40. The system of claim 39, wherein the initial data set comprises one or more initial triggers.

41. The system of claim 39, wherein the initial dataset does not include initial triggers.

42. The system of claim 39, wherein the initial data set comprises one or more initial boosts.

43. The system of claim 39, wherein the initial dataset does not include initial boosts.

44.-46. (canceled)

47. The system of claim 1, wherein the one or more thresholds over time represents a change greater than about 30%.
